SEQUENCE LISTING (3 sequences)

SEQ ID NO: 1 - protein, 17 aa, Artificial Sequence
  Synthesized
  MOD_RES 9: Xaa is Lys, in which both of the alpha- and epsilon-amino groups thereof are associated with amide bond formation, and the lysyl carboxyl group is protected.
KGVSLSYRXRYSLSVGK

SEQ ID NO: 2 - protein, 19 aa, Artificial Sequence
  Synthesized
  MOD_RES 10: Xaa is Lys, in which both of the alpha- and epsilon-amino groups thereof are associated with amide bond formation to the adjacent cysteine amino acid residues.
KGVSLSYRCXCRYSLSVGK

SEQ ID NO: 3 - protein, 9 aa, Artificial Sequence
  Synthesized
KGVSLSYRC

(19) United States
(12) Patent Application Publication
(10) Pub. No.: US 2019/0233524 A1
(43) Pub. Date: Aug. 1, 2019

(54) THERAPEUTIC COMBINATION AND METHOD FOR TREATING CANCER

(71) Applicant: TCM BIOTECH INTERNATIONAL CORP., New Taipei (TW)

(72) Inventors: YA-CHUN WANG, New Taipei (TW); JEN-YAU CHEN, New Taipei (TW)

(21) Appl. No.: 16/132,339

(22) Filed: Sep. 14, 2018

Related U.S. Application Data
(60) Provisional application No. 62/559,728, filed on Sep. 18, 2017.

(51) Int. Cl. C07K 16/28; A61P 35/00; A61K 31/7105; C12N 15/113; A61K 39/00; A61K 35/17; A61K 9/00

(52) U.S. Cl. CPC C07K 16/2866; A61P 35/00; A61K 31/7105; A61K 9/0019; A61K 39/0011; A61K 35/17; C12N 15/113

(57) ABSTRACT

A therapeutic combination for treating cancer in a subject having a tumor in provided. The therapeutic combination includes an immunotherapeutics for treating the cancer, and a peptide having one of SEQ ID NOs. 1-3 and being capable of selectively binding to CXC chemokine receptor 4 (CXCR4). When the peptide of the therapeutic combination binds to CXCR4, an immune microenvironment of the tumor is modulated and/or accessibility of immune cells to the tumor is regulated. A method for treating cancer using the therapeutic combination is also provided.

### CROSS-REFERENCE TO RELATED APPLICATION

The application claims the benefit of U.S. provisional patent application No. 62/559,728, filed on Sep. 18, 2017, the entirety of which is incorporated herein by reference.

### FIELD OF THE DISCLOSURE

The present disclosure relates to a therapeutic combination, and more particularly, using a peptide that selectively binds to CXCR4 in combination with one or more immunotherapeutics for treatment of cancer or viral infection.

### BACKGROUND OF THE DISCLOSURE

In addition to chemotherapy, radiation, and surgery, immunotherapy has been recognized as the fourth pillar of cancer treatment. Existing cancer immunotherapeutics are mainly monoclonal antibodies (mAbs) that block protein-protein interactions between T cell checkpoint receptors and their cognate ligands. Clinically approved mAbs include T cell checkpoint inhibitory antibodies ipilimumab (Yervoy® by Bristol-Myers Squibb), pembrolizumab (Keytruda® by Merck), nivolumab (Opdivo® by Bristol-Myers Squibb), atezolizumab (Tecentriq® by Roche/Genetech), avelumab (Bavencio® by EMD Serono), and durvalumab (Imfinzi® by AstraZeneca).

Specifically, ipilimumab is a fully human IgG1 mAb that directly binds to the cytotoxic T lymphocyte-associated antigen 4 (CTLA4) receptor protein to block a critical inhibitory signal for activated T cells. Pembrolizumab and nivolumab are humanized anti-PD-1 monoclonal antibodies (mAbs) that block ligand engagement to programmed cell death protein 1 (PD-1), thus interfering with T cell signaling and cell death. Likewise, atezolizumab, avelumab, and durvalumab are humanized anti-PD-L1 mAbs that achieve similar functions by inhibiting receptor engagement to programmed cell death protein ligand 1 (PD-L1).

Meanwhile, genetically engineered autologous T cell therapies take a more direct approach on T cell activation and cancer cell targeting, and have also demonstrated significant clinical responses in haematological cancers. In such therapies, chimeric antigen receptors (CARs) or cancer target-specific T cell receptors (TCRs) are transduced and expressed in a patient's T cells ex vivo to render the cells tumor specificity before reinfusing the T cells into the patient.

However, due to the various steps required for eliciting an effective T cell-mediated antitumor immune response or activating the associated immunosuppressive mechanisms, existing single-agent immunotherapies (or immune monotherapies) often result in limited immune response or are unable to overcome immune suppression at the tumor microenvironment, leading to immune escape, continued tumor growth and hence poor treatment efficacy for the vast majority of cancer patients.

### BRIEF SUMMARY OF THE DISCLOSURE

An objective of the present disclosure is to provide an adjuvant enhanced immunotherapy that promotes effective antitumor immune response.

Another objective of the present disclosure is to provide an immunotherapy adjuvant that regulates immunosuppressive tumor microenvironment.

An embodiment of the present disclosure provides a therapeutic combination for treating cancer in a subject having a tumor. The therapeutic combination includes an immunotherapeutics for treating the cancer, and a peptide having one of SEQ ID NOs. 1-3 and being capable of selectively binding to CXC chemokine receptor 4 (CXCR4).

Preferably, the immunotherapeutics selectively targets CTLA-4, PD-1, PD-L1, TIM-3, LAG-3, B7-1, B7-H3, NKG2A, KIR, BTLA, VISTA/PD-1H, TIGIT, CD96, OX40, CD28, ICOS, HVEM, 41BB, CD40L, CD137, GITR, CD27, CD30, DNAM-1, CD28H or coreceptors thereof.

Preferably, the immunotherapeutics is an antibody, a vaccine, a cytokine, a protein, a peptide, an expression vector encoding the protein or the peptide, a small molecule, an RNAi, or an aptamer.

Preferably, the immunotherapeutics is autologous immune cells, tumor-specific autologous T cells, T-cell receptor (TCR)-engineered T cells, or chimeric antigen receptor T (CAR-T) cells.

Preferably, an immune microenvironment of the tumor is modulated when the peptide binds to CXCR4.

Preferably, accessibility of immune cells to the tumor is regulated when the peptide binds to CXCR4.

Preferably, the immune cells include CD45+ cells, CD3+ T cells, CD4+CD8− T cells, CD4−CD8+ T cells, T-reg cells, NK cells, NKT cells, macrophages, granulocytes, and/or monocytes.

Preferably, the cancer treated by the therapeutic combination is breast cancer, colon cancer, lung cancer, pancreatic cancer, prostate cancer, kidney cancer, liver cancer, lymphoma or melanoma.

Another embodiment of the present disclosure provides a method for treating cancer in a subject having a tumor. The method includes a step of: administering to the subject the aforementioned therapeutic combination.

Preferably, the peptide is administered to the subject intravenously, subcutaneously, or intraperitoneally.

In sum, according to various embodiments of the present disclosure, the peptide having one of SEQ ID Nos. 1-3 (e.g., PTX-9908) is complementary to and synergistic with immunotherapeutics by allowing modulation of tumor immune microenvironment and/or regulation of accessibility of immune cells to the tumor, therefore improving efficacy of the immunotherapy.

### BRIEF DESCRIPTION OF THE DRAWINGS

The accompanying drawings illustrate one or more embodiments of the present invention and, together with the written description, explain the principles of the present invention. Wherever possible, the same reference numbers are used throughout the drawings to refer to the same or like elements of an embodiment.

FIG. 1 is a schematic illustration of the mechanism of PTX-9908 in modulation of immunosuppressed tumor microenvironment for enhancing efficacy of the combined immunotherapies in accordance with an exemplary embodiment of the present disclosure;

FIG. 2A is an experiment result showing the differences in mean tumor volume in MC38 xenograft mouse models treated with or without the therapeutic combination in accordance with an exemplary embodiment of the present disclosure;

FIG. 2B is an experiment result showing the difference in tumor volume inhibition in the MC38 xenograft mouse models treated with or without the therapeutic combination in accordance with an exemplary embodiment of the present disclosure;

FIG. 3A is an experiment result showing the differences in tumor weight in the MC38 xenograft mouse models treated with or without the therapeutic combination in accordance with an exemplary embodiment of the present disclosure;

FIG. 3B is an experiment result showing the differences in tumor growth inhibition (TGI) in the MC38 xenograft mouse models treated with or without the therapeutic combination in accordance with an exemplary embodiment of the present disclosure;

FIG. 4 is an experiment result showing the consistency in body weight in the MC38 xenograft mouse models treated with or without the therapeutic combination in accordance with an exemplary embodiment of the present disclosure;

FIG. 5 is an experiment result showing the differences in immune cell profile in tumors collected from the MC38 xenograft mouse models treated with or without the therapeutic combination in accordance with an exemplary embodiment of the present disclosure;

FIG. 6A is an experiment result showing the differences in mean tumor volume in EMT-6 xenograft mouse models treated with or without the therapeutic combination in accordance with an exemplary embodiment of the present disclosure;

FIG. 6B is an experiment result showing the difference in tumor volume inhibition in the EMT-6 xenograft mouse models treated with or without the therapeutic combination in accordance with an exemplary embodiment of the present disclosure;

FIG. 7A is an experiment result showing the differences in tumor weight in the EMT-6 xenograft mouse models treated with or without the therapeutic combination in accordance with an exemplary embodiment of the present disclosure;

FIG. 7B is an experiment result showing the differences in tumor growth inhibition (TGI) in the EMT-6 xenograft mouse models treated with or without the therapeutic combination in accordance with an exemplary embodiment of the present disclosure;

FIG. 8 is an experiment result showing the consistency in body weight in the EMT-6 xenograft mouse models treated with or without the therapeutic combination in accordance with an exemplary embodiment of the present disclosure;

FIG. 9 is an experiment result showing the differences in immune cell profile in tumors collected from the EMT-6 xenograft mouse models treated with or without the therapeutic combination in accordance with an exemplary embodiment of the present disclosure;

FIG. 10A is an experiment result showing the differences in mean tumor volume in LL/2 xenograft mouse models treated with or without the therapeutic combination in accordance with an exemplary embodiment of the present disclosure;

FIG. 10B is an experiment result showing the difference in tumor volume inhibition in the LL/2 xenograft mouse models treated with or without the therapeutic combination in accordance with an exemplary embodiment of the present disclosure;

FIG. 11 is an experiment result showing the consistency in body weight in the LL/2 xenograft mouse models treated with or without the therapeutic combination in accordance with an exemplary embodiment of the present disclosure; and

FIG. 12 is an experiment result showing the differences in immune cell profile in tumors collected from the LL/2 xenograft mouse models treated with or without the therapeutic combination in accordance with an exemplary embodiment of the present disclosure.

In accordance with common practice, the various described features are not drawn to scale and are drawn to emphasize features relevant to the present disclosure. Like reference characters denote like elements throughout the figures and text.

### DETAILED DESCRIPTION OF THE PREFERRED EMBODIMENTS

The present invention will now be described more fully hereinafter with reference to the accompanying drawings illustrating various exemplary embodiments of the invention. The present invention may, however, be embodied in many different forms and should not be construed as limited to the embodiments set forth herein. Rather, these embodiments are provided so that this disclosure will be thorough and complete, and will fully convey the scope of the disclosure to those skilled in the art. Like reference numerals refer to like elements throughout.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” or “includes” and/or “including” or “has” and/or “having” when used herein, specify the presence of stated features, regions, integers, steps, operations, elements, and/or components, but do not preclude the presence or addition of one or more other features, regions, integers, steps, operations, elements, components, and/or groups thereof.

It will be understood that the terms “and/or” and “at least one” include any and all combinations of one or more of the associated listed items. It will also be understood that, although the terms first, second, third etc. may be used herein to describe various elements, components, regions, parts and/or sections, these elements, components, regions, parts and/or sections should not be limited by these terms. These terms are only used to distinguish one element, component, region, part or section from another element, component, region, layer or section. Thus, a first element, component, region, part or section discussed below could be termed a second element, component, region, layer or section without departing from the teachings of the present disclosure.

As used herein, the terms “PTX-9908” (also known as CTCE-9908) refers to a small analog peptide having any one of SEQ ID NOs: 1-3 and consisting of a monomer or dimer of a partial sequence of stromal cell derived factor one (SDF-1; also known as CXCL12). PTX-9908 is a CXC chemokine receptor 4 (CXCR4) antagonist and blocks SDF-1 from binding to CXCR4. CXCR4 is a seven transmembrane G1-coupled protein and is expressed in a wide range of immune cells, including T cells, B cells, monocytes, polymorphonuclear cells (PMNCs), immature dendritic cells (DCs), as well as in solid and hematopoietic malignancies. The SDF-1/CXCR4 pathway has been shown to associate with immune cell mobilization, cancer metastasis, and HIV entry into host cells.

PTX-9908 described in the present disclosure can be obtained according to methods described in U.S. Pat. No. 7,423,011. In the various embodiments of the present disclosure, PTX-9908 may be a substantially purified peptide, a purified peptide fragment, a modified peptide, a modified peptide fragment, an analog of PTX-9908.

As used herein, the term “immunotherapeutics” may include, but are not limited to, monoclonal antibodies, vaccines, recombinant cytokines, affinity proteins or engineered non-antibody peptides, expression vectors encoding the affinity proteins or engineered non-antibody peptides, small molecules, RNAi, and/or aptamers that target cytotoxic T lymphocyte-associated antigen 4 (CTLA-4), programmed cell death-1 (PD-1), programmed cell death ligand-1 (PD-L1), T cell immunoglobulin and mucin domain 3 (TIM-3), lymphocyte-activation gene 3 (LAG-3), cluster of differentiation 80 (CD80; also known as B7-1), cluster of differentiation 276 (CD276; also known as B7-H3), cluster of differentiation 94 (CD94; also known as NKG2A), killer-cell immunoglobulin-like receptor (KIR), B- and T-lymphocyte attenuator (BTLA), V-domain Ig suppressor of T cell activation (VISTA), programmed cell death-1 homolog (PD-1H), T cell immunoglobulin and immunoreceptor tyrosine-based inhibitory motif domain (TIGIT), cluster of differentiation 96 (CD96), cluster of differentiation 134 (CD134; also known as OX40), cluster of differentiation 28 (CD28), inducible T-cell costimulator (ICOS), herpesvirus entry mediator (HVEM), cluster of differentiation 137 (CD137; also known as 41BB), cluster of differentiation 154 (CD154; also known as CD40L), glucocorticoid-induced TNFR-related protein (GITR), cluster of differentiation 27 (CD27), cluster of differentiation 30 (CD30), DNAX accessory molecule-1 (DNAM-1), cluster of differentiation 28 homolog (CD28H) or other immune cell receptors and their coreceptors.

The term “immunotherapeutics” described herein may also referred to “immunotherapeutic cells,” such as cytokine-induced killer (CIK) cells, natural killer (NK) cells, dendritic cells (DC), DC-CIK cells, gammadelta T cells, autologous immune cells, genetically engineered chimeric antigen receptor T (CAR-T) cells, genetically engineered T-cell receptor (TCR) T cells, tumor-specific autologous T cells, autologous tumor infiltrating lymphocytes (TIL), and/or genetically re-directed peripheral blood mononuclear cells that are used to be transfused into a subject in immune cell therapies.

As used herein, the term “immune cells” may include CD45+ cells, CD3+ T cells, CD4+CD8− T cells, CD4−CD8+ T cells, T-reg cells, NK cells, natural killer T (NKT) cells, macrophages, granulocytes, monocytes, CIK cells, dendritic cells, DC-CIK cells, gammadelta T cells, genetically engineered CAR-T cells, genetically engineered TCR T cells, tumor-specific autologous T cells, autologous TIL, and/or genetically re-directed peripheral blood mononuclear cells.

As used herein, the term “treating” or “treatment” encompasses both disease-modifying treatment and symptomatic treatment, either of which may be therapeutic (i.e., after the onset of symptoms, in order to reduce the severity and/or duration of symptoms). Treatment methods provided herein include, in general, administration to a subject an effective amount of one or more small molecules, peptides, antibodies, RNAi, or aptamers provided herein. Suitable subjects include patients suffering from or susceptible to a disorder or disease identified herein. Typical patients for treatment as described herein include mammals, particularly primates, especially humans. Other suitable patients include domesticated companion animals, such as a dog, cat, horse, and the like, or a livestock animal such as cattle, pig, sheep and the like.

In an aspect of the present disclosure, PTX-9908 is used in combination with one or more immunotherapeutics to treat or produce medicaments to treat, a variety of cancers. Such variety of cancers include, but are not limited to, unresectable or metastatic (advanced) melanoma, metastatic non-small cell lung cancer (NSCLC), recurrent or metastatic squamous cell carcinoma of the head and neck (SCCHN), classical Hodgkin lymphoma (cHL), locally advanced or metastatic urothelial carcinoma, solid tumor cancers expressing biomarker microsatellite instability-high (MSI-H) or with mismatch repair deficiency (dMMR), metastatic renal cell carcinoma, hepatocellular carcinoma (HCC), metastatic Merkel cell carcinoma (MCC), and other types of carcinoma of the skin, lung, kidney, bladder, head and neck, liver, breast and other organs of the body, as well as leukemia, multiple myeloma, and other types of cancers of the circulatory systems.

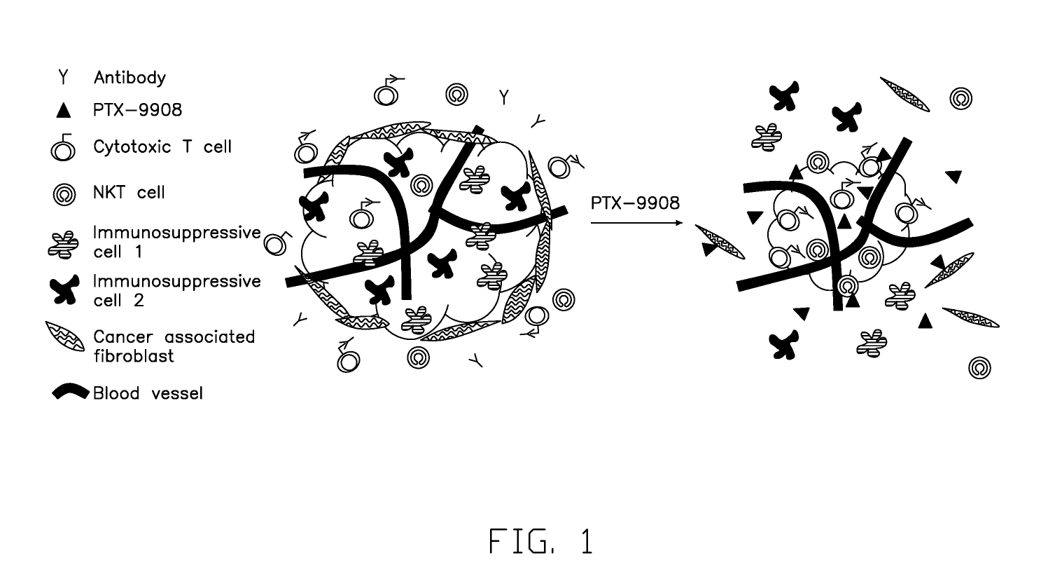

In some embodiments, tumor immune microenvironment may be modulated when PTX-9908 binds to CXCR4. For example, as exemplarily illustrated in FIG. 1, binding of PTX-9908 to CXCR4 may result in modulation of immunosuppressed tumor microenvironment, therefore allowing the combined immunotherapeutics (e.g., antibodies 1) to exert their full therapeutic potential.

In other embodiments, accessibility of immune cells to the site of tumor may be regulated when PTX-9908 binds to CXCR4. Specifically, binding of PTX-9908 to CXCR4 may also result in modulated mobilization or infiltration of immune cells at the tumor microenvironment, and/or, as exemplified in FIG. 1, cause loosening of the barrier formed by cancer associated fibroblasts. Therefore, cytotoxic immune cells (also known as immune effector cells; e.g., CD3+ T cells, CD8+ T cells, NK cells, and NKT cells) or immunotherapeutic cells are allowed to access and eliminate the tumor, and/or suppressive immune cells (e.g., monocytes, granulocytes, regulatory T (T-reg) cells) at the tumor microenvironment are reduced.

In another aspect of the present disclosure, PTX-9908 is used in combination with one or more immunotherapeutics to treat or produce medicaments to treat, a variety of viral infections. Such viral infections include, but are not limited to, infections with human immunodeficiency virus (HIV), human papillomavirus (HPV), Epstein-Barr (EBV), cytomegalovirus (CMV), human herpesvirus (HHV), Varicella zoster virus (VZV), hepatitis virus, measles virus, adenovirus, or other viruses that may cause persistent infection in the host.

In some embodiments, immune microenvironment at the site of viral infection may be regulated when PTX-9908 binds to CXCR4. For example, binding of PTX-9908 to CXCR4 may result in activation of immunosuppressed microenvironment at the infection site, therefore allowing the combined immunotherapeutics (e.g., antibodies) to exert their full therapeutic potential.

In other embodiments, accessibility of immune cells to the site of viral infection may be regulated when PTX-9908 binds to CXCR4. For example, binding of PTX-9908 to CXCR4 may result in increased mobilization or infiltration of immune cells to the infection site, therefore allowing activated immune cells or immunotherapeutic cells to access and eliminate the virus.

Yet another aspect of the present disclosure pertains to methods for treating a subject suffering from or susceptible to one or more of a variety of cancers. Such cancers may include unresectable or metastatic (advanced) melanoma, metastatic non-small cell lung cancer (NSCLC), recurrent or metastatic squamous cell carcinoma of the head and neck (SCCHN), classical Hodgkin lymphoma (cHL), locally advanced or metastatic urothelial carcinoma, solid tumor cancers expressing biomarker microsatellite instability-high (MSI-H) or with mismatch repair deficiency (dMMR), metastatic renal cell carcinoma, hepatocellular carcinoma (HCC), metastatic Merkel cell carcinoma (MCC), and other types of carcinoma of the skin, lung, kidney, bladder, head and neck, liver, breast and other organs of the body, as well as leukemia, multiple myeloma, and other types of cancers of the circulatory systems.

In an embodiment, the method includes administering to the subject PTX-9908 in combination with one or more immunotherapeutics. Preferably, PTX-9908 is administered to the subject intravenously, subcutaneously, or intraperitoneally. The administered PTX-9908 is preferably in a therapeutically effective amount sufficient to modulate immunosuppressed tumor microenvironment and/or regulate accessibility of immune cells to the site of tumor. In other words, PTX-9908 is administered to the subject in an amount sufficient to produce synergistic effect with the combined immunotherapy for treatment of cancer.

Still another aspect of the present disclosure pertains to methods for treating a subject suffering from or susceptible to one or more viral infections. Such viral infections may include infections with human immunodeficiency virus (HIV), human papillomavirus (HPV), Epstein-Barr (EBV), cytomegalovirus (CMV), human herpesvirus (HHV), Varicella zoster virus (VZV), hepatitis virus, measles virus, adenovirus, or other viruses that may cause persistent infection in the host.

In an embodiment, the method includes administering to the subject PTX-9908 in combination with one or more immunotherapeutics. Preferably, PTX-9908 is administered to the subject intravenously, subcutaneously, or intraperitoneally. The administered PTX-9908 is preferably in a therapeutically effective amount sufficient to modulate immunosuppressed microenvironment at the site of viral infection and/or regulate accessibility of immune cells to the infection site. In other words, PTX-9908 is administered to the subject in an amount sufficient to produce synergistic effect with the combined immunotherapy for treatment of viral infection.

A “therapeutically effective amount” refers to an amount effective, at dosages and for periods of time necessary, to achieve the desired therapeutic result, such as suppression or inhibition of tumor growth or viral infection at the site of infection or in the circulatory system. A therapeutically effective amount of PTX-9908 may vary according to factors such as the disease stage, age, gender, and weight of the subject, and the ability of PTX-9908 to elicit a desired response in the subject. Dosage regimens may be adjusted to provide the optimum therapeutic response. A therapeutically effective amount is also one in which any toxic or detrimental effects of PTX-9908 are outweighed by the therapeutically beneficial effects.

It is to be noted that dosages of PTX-9908 may vary with the severity of the condition to be alleviated. It is to be further understood that for any particular subject, specific dosage regimens should be adjusted over time according to the individual need and the professional judgment of the person administering or supervising the administration of the therapeutic combination.

To facilitate administration of PTX-9908 into the subject, PTX-9908 is preferably combined with a pharmaceutically acceptable carrier or excipient, which may include any and all solvents, dispersion media, coatings, antibacterial and antifungal agents, isotonic and absorption delaying agents, and the like that are physiologically compatible. In one embodiment, the carrier is suitable for parenteral administration. Alternatively, the carrier can be suitable for intravenous, subcutaneous, intraperitoneal, intramuscular, sublingual or oral administration. Pharmaceutically acceptable carriers include sterile aqueous solutions or dispersions and sterile powders for the extemporaneous preparation of sterile injectable solutions or dispersion.

PTX-9908 may be formulated as a solution, microemulsion, liposome, or other ordered structure suitable to high drug concentration. The carrier may be a solvent or dispersion medium containing, for example, water, ethanol, polyol (for example, glycerol, propylene glycol, and liquid polyethylene glycol, and the like), and suitable mixtures thereof. The proper fluidity may be maintained, for example, by the use of a medium such as lecithin, by the maintenance of the required particle size in the case of dispersion and by the use of surfactants. In many cases, it is preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. The injectable compositions may be formulated with one or more additional compounds that enhance the solubility of PTX-9908.

Moreover, PTX-9908 may be administered in a time release formulation, for example in a composition which includes a slow release polymer, or may be prepared with carriers that would protect PTX-9908 against rapid release, such as a controlled release formulation, including implants and microencapsulated delivery systems. Biodegradable, biocompatible polymers may be used, such as ethylene vinyl acetate, polyanhydrides, polyglycolic acid, collagen, polyorthoesters, polylactic acid and polylactic, polyactic-polyglycolic copolymers (PLG). Many methods for the preparation of such formulations are patented or generally known to those skilled in the art.

Sterile injectable solutions can be prepared by incorporating PTX-9908 in the required amount in an appropriate solvent with one or a combination of ingredients enumerated above, as required, followed by filtered sterilization. Generally, dispersions can be prepared by incorporating PTX-9908 into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of PTX-9908 plus any additional desired ingredient from a previously sterile-filtered solution thereof.

PTX-9908 in the embodiments of the present disclosure may be modified to alter the specific properties of the peptides while retaining its abilities to modulate immune microenvironment or regulate accessibility of immune cells. For example, PTX-9908 may be modified to alter their pharmacokinetic properties, such as in vivo stability or half-life. PTX-9908 may also be modified to label the peptides with one or more detectable substance, such as various enzymes, prosthetic groups, fluorescent materials, luminescent materials and radioactive materials. Further, PTX-9908 may also be modified to be coupled to one or more functional moiety for additional or enhanced therapeutic properties.

In an alternative modification, PTX-9908 in the embodiments of the present disclosure may be prepared in a “prodrug” form, wherein the peptides per se do not modulate immune microenvironment or regulate accessibility of immune cells, but rather are capable of being transformed, upon metabolism in vivo, into immunologically active PTX-9908.

Efficacy Evaluation—I

MC38 human colon cancer cells were inoculated into 30 7-9 week-old C57BL/6 female mice, and treatment was initiated when tumors in the mice reached a mean volume of approximately 80-120 mm³ (or around 100 mm³). The mice were randomly divided into three groups (i.e., 10 mice per group). Group 1 was intraperitoneally administered with phosphate buffered saline (PBS) biweekly for 5 doses. Group 2 was intraperitoneally administered with 10 mg/kg of anti-PD-1 antibody in PBS, biweekly for 5 doses. Group 3 was intraperitoneally administered with 10 mg/kg of anti-PD-1 antibody in PBS, biweekly for 5 doses, and 25 mg/kg of PTX-9908 in a 5-days-on-2-days-off schedule for 13 doses. The study was terminated when the mean tumor volume in Group I reached 2,000 mm³.

In the study, tumor volume is expressed in mm³ using the formula: V=(L×W×W)/2; wherein V means tumor volume, L means tumor length (i.e., the longest tumor dimension) and W means tumor width (i.e., the longest tumor dimension perpendicular to L). Statistical analysis of differences in mean tumor volume among the groups was conducted by Independent-Samples T Test using the data collected. P-values were rounded to three decimal places, with the exception that raw P-values less than 0.001 were stated as P<0.001. All tests were two-sided.

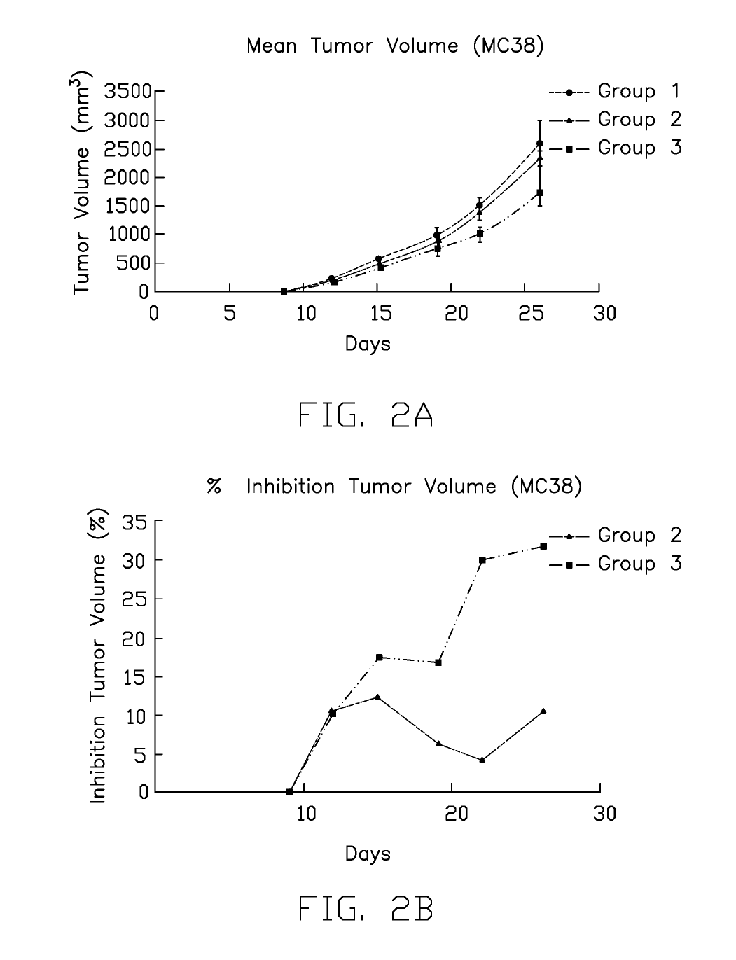

As shown in FIG. 2A, tumor growth curves (i.e., time-dependent change in mean tumor volume) of the three groups show a reduction in mean tumor volume in Group 3. Mean percentage of tumor volume inhibition was also calculated from the measured tumor volume according to the formula: mean % inhibition=(mean(C)−mean(T))/mean(C)×100%; wherein T means current group value, and C means control group value. As shown in FIG. 2B, Group 3 also exhibited significantly greater tumor volume inhibition than Group 2.

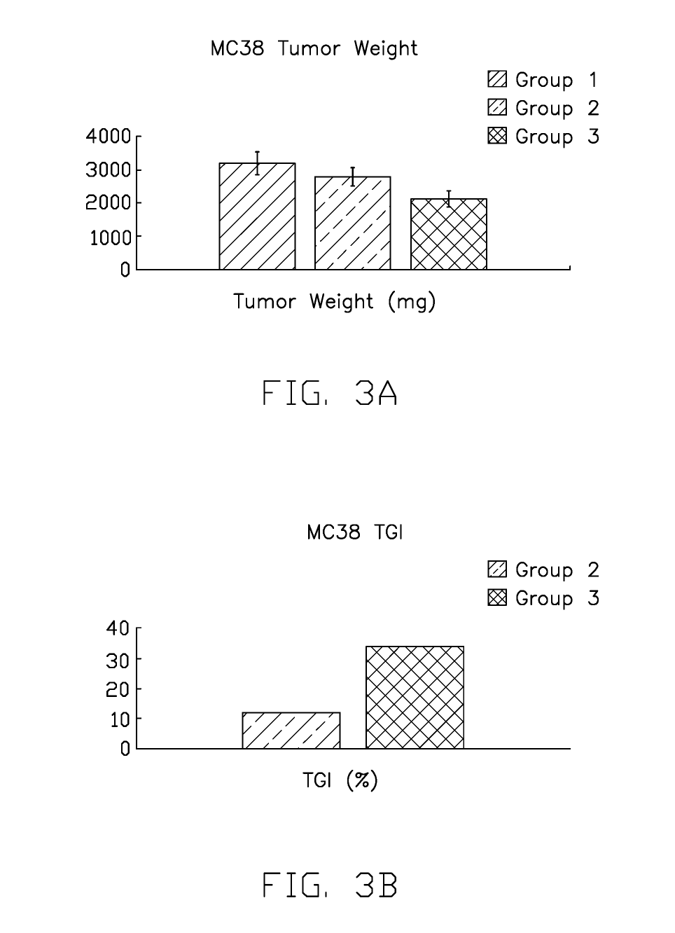

Tumor weight was also measured at the end of the study. As shown in FIG. 3A, Group 3 exhibited a 24.6% reduction in tumor weight as compared with Group 2. Tumor growth inhibition (TGI) was also calculated from the measured tumor weight according to the formula: mean % inhibition=(mean(C)−mean(T))/mean(C)×100%; wherein T means current group value, and C means control group value. As shown in FIG. 3B, Group 3 also exhibited significantly greater TGI than Group 2.

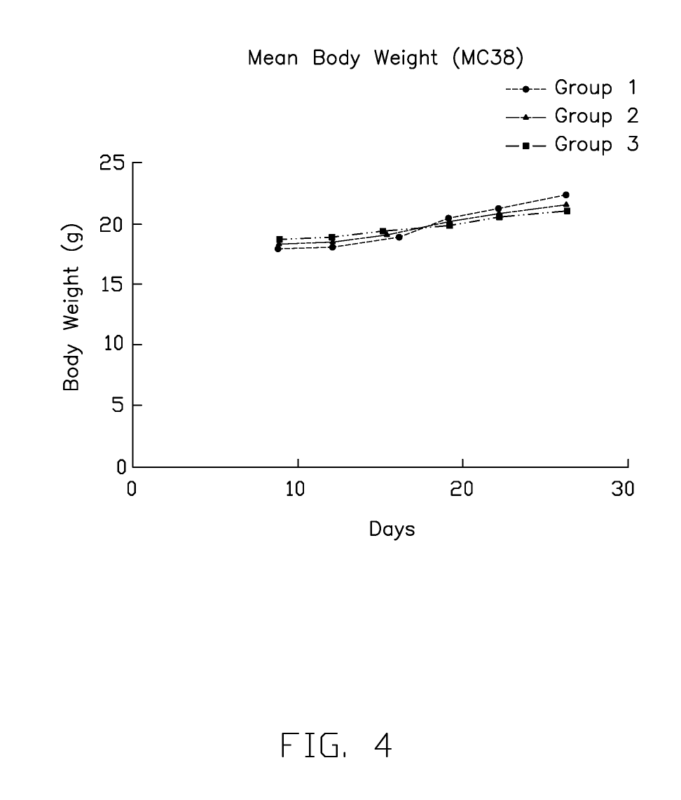

Body weight of the mice was also monitored during the course of the study. As shown in FIG. 4, no adverse effect on body weight was observed in Group 3.

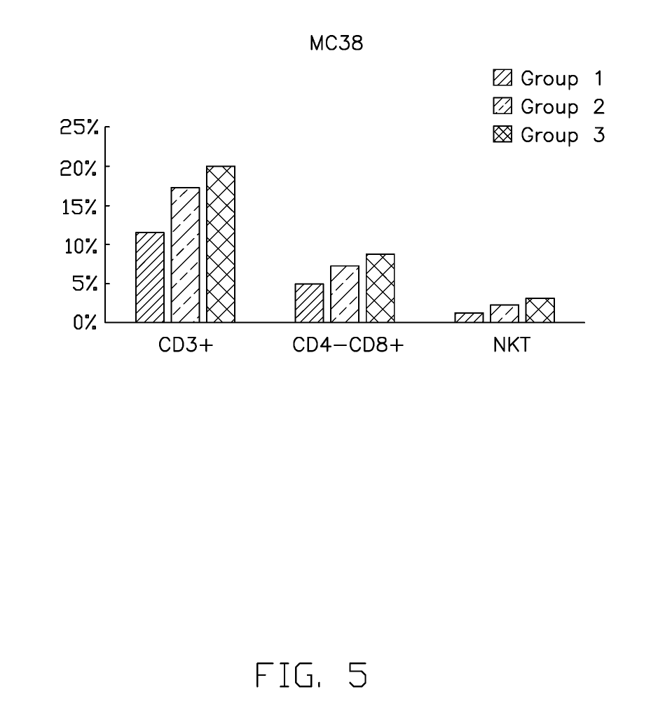

Further, as shown in FIG. 5, flow cytometric analysis of live cells in the tumor revealed that, as compared with the other groups, Group 3 contains higher percentages of CD3+ T cells, CD4−CD8+ T cells and NKT cells over the CD45+ cell population, suggesting upregulation of accessibility of cytotoxic immune cells to the tumor microenvironment.

The results shown in FIGS. 2-5 unambiguously demonstrate the synergistic effect of PTX-9908 in anti-PD-1 treatment for colon cancer.

Efficacy Evaluation—II

EMT-6 human breast cancer cells were inoculated into 30 7-9 week-old BALB/C female mice, and treatment was initiated when tumors in the mice reached a mean volume of approximately 80-120 mm³ (or around 100 mm³). The mice were randomly divided into three groups. Group 1 was intraperitoneally administered with phosphate buffered saline (PBS) biweekly for 6 doses. Group 2 was intraperitoneally administered with 10 mg/kg of anti-PD-1 antibody in PBS, biweekly for 6 doses. Group 3 was intraperitoneally administered with 10 mg/kg of anti-PD-1 antibody in PBS, biweekly for 6 doses, and 25 mg/kg of PTX-9908 in a 5-days-on-2-days-off schedule for 15 doses. The study was terminated when the mean tumor volume in Group I reached 2,000 mm³.

In the study, tumor volume is expressed in mm³ using the formula: V=(L×W×W)/2; wherein V means tumor volume, L means tumor length (i.e., the longest tumor dimension) and W means tumor width (i.e., the longest tumor dimension perpendicular to L). Statistical analysis of differences in mean tumor volume among the groups was conducted by Independent-Samples T Test using the data collected. P-values were rounded to three decimal places, with the exception that raw P-values less than 0.001 were stated as P<0.001. All tests were two-sided.

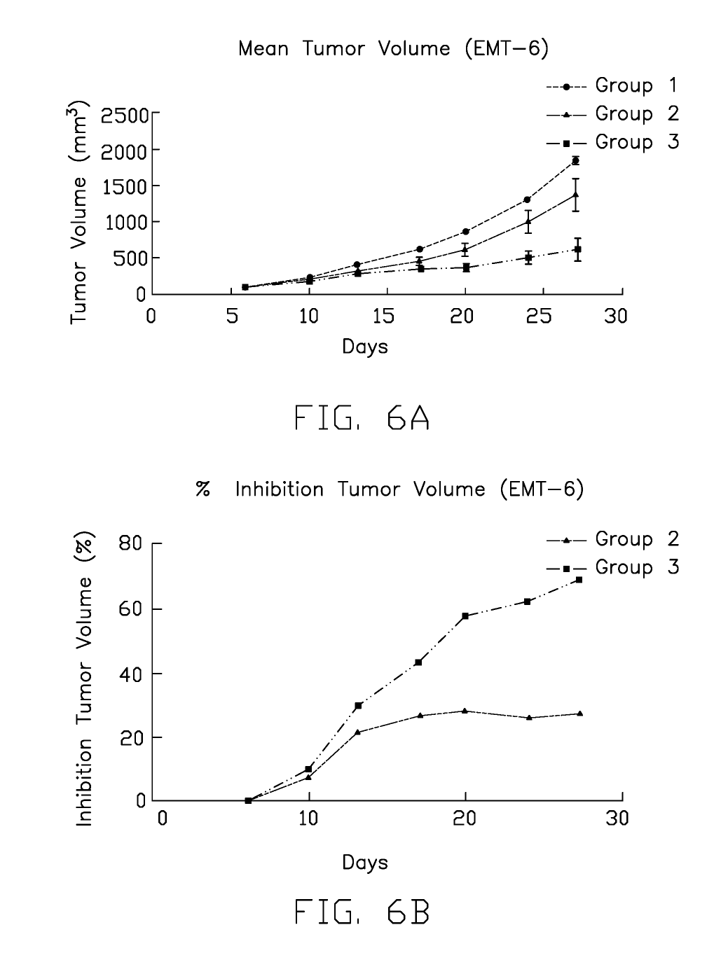

As shown in FIG. 6A, tumor growth curves (i.e., time-dependent change in mean tumor volume) of the three groups show a reduction in mean tumor volume in Group 3. Mean percentage of tumor volume inhibition was also calculated from the measured tumor volume according to the formula: mean % inhibition=(mean(C)−mean(T))/mean(C)×100%; wherein T means current group value, and C means control group value. As shown in FIG. 6B, Group 3 also exhibited significantly greater tumor volume inhibition than Group 2.

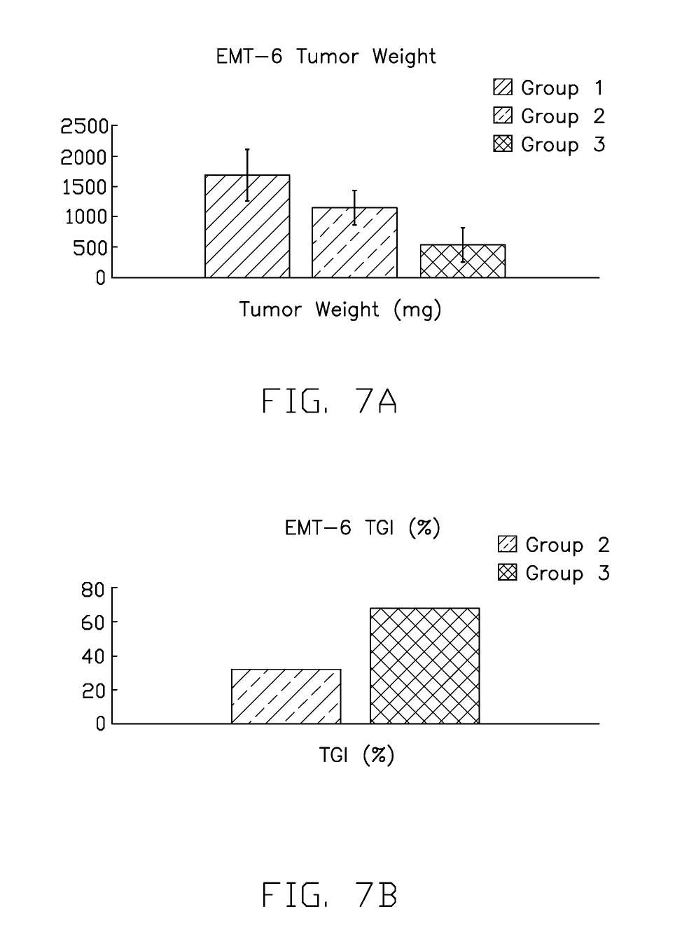

Tumor weight was also measured at the end of the study. As shown in FIG. 7A, Group 3 exhibited a 53.7% reduction in tumor weight as compared with the Group 2. Tumor growth inhibition (TGI) was also calculated from the measured tumor weight according to the formula: mean % inhibition=(mean(C)−mean(T))/mean(C)×100%; wherein T means current group value, and C means control group value. As shown in FIG. 7B, Group 3 also exhibited significantly greater TGI than Group 2.

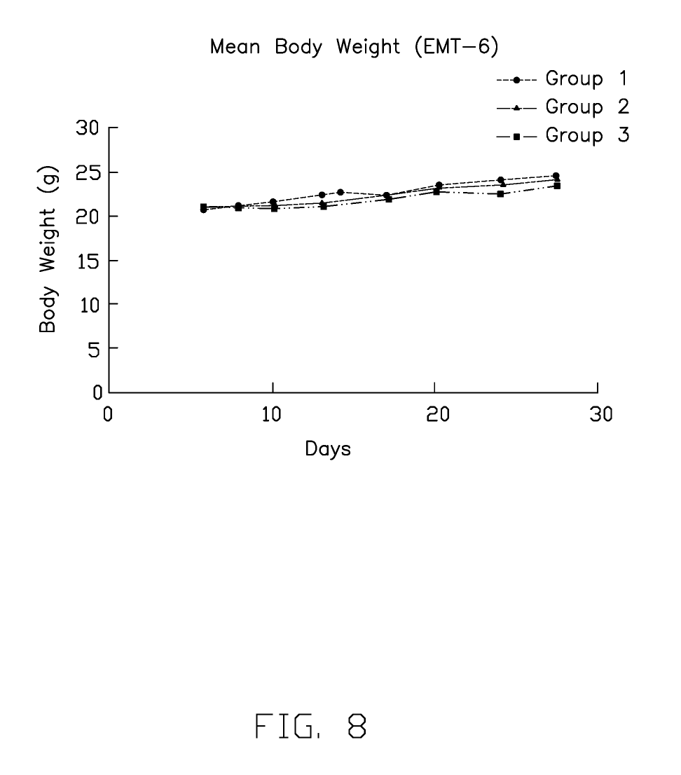

Body weight of the mice was also monitored during the course of the study. As shown in FIG. 8, no adverse effect on body weight was observed in Group 3.

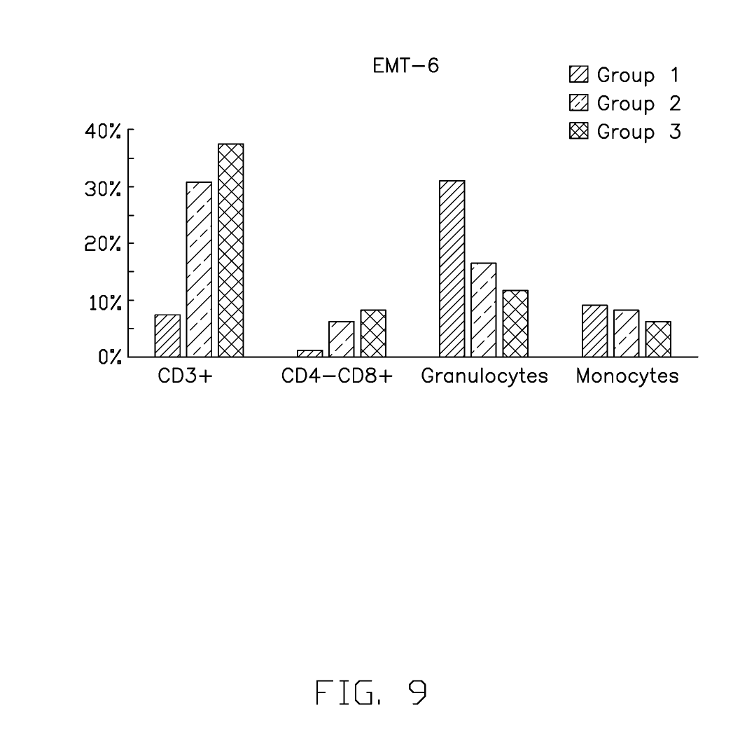

Further, as shown in FIG. 9, flow cytometric analysis of live cells in the tumor revealed that, as compared with the other groups, Group 3 contains higher percentages of CD3+ T cells and CD4−CD8+ T cells and lower percentages of granulocytes and monocytes over the CD45+ cell population. As the presence of granulocytes and monocytes in the tumor microenvironment has been known to negatively modulate the anti-tumor effects mediated by anti-PD-1 antibody, the results suggest upregulation of accessibility of cytotoxic immune cells and downregulation of accessibility of suppressive immune cells to the tumor microenvironment.

The results shown in FIGS. 6-9 unambiguously demonstrate the synergistic effect of PTX-9908 in anti-PD-1 treatment for breast cancer.

Efficacy Evaluation—III

LL/2 human lung cancer cells were inoculated into 30 7-9 week-old CS7BL/6 female mice, and treatment was initiated when tumors in the mice reached a mean volume of approximately 80-120 mm³ (or around 100 mm³). The mice were randomly divided into three groups. Group 1 was intraperitoneally administered with phosphate buffered saline (PBS) biweekly for 5 doses. Group 2 was intraperitoneally administered with 10 mg/kg of anti-PD-1 antibody in PBS, biweekly for 5 doses. Group 3 was intraperitoneally administered with 10 mg/kg of anti-PD-1 antibody in PBS, biweekly for 5 doses, and 25 mg/kg of PTX-9908 in a 5-days-on-2-days-off schedule for 13 doses. The study was terminated when the mean tumor volume in Group 1 reached 2,000 mm³.

In the study, tumor volume is expressed in mm³ using the formula: V=(L×W×W)/2; wherein V means tumor volume, L means tumor length (i.e., the longest tumor dimension) and W means tumor width (i.e., the longest tumor dimension perpendicular to L). Statistical analysis of differences in mean tumor volume among the groups was conducted by Independent-Samples T Test using the data collected. P-values were rounded to three decimal places, with the exception that raw P-values less than 0.001 were stated as P<0.001. All tests were two-sided.

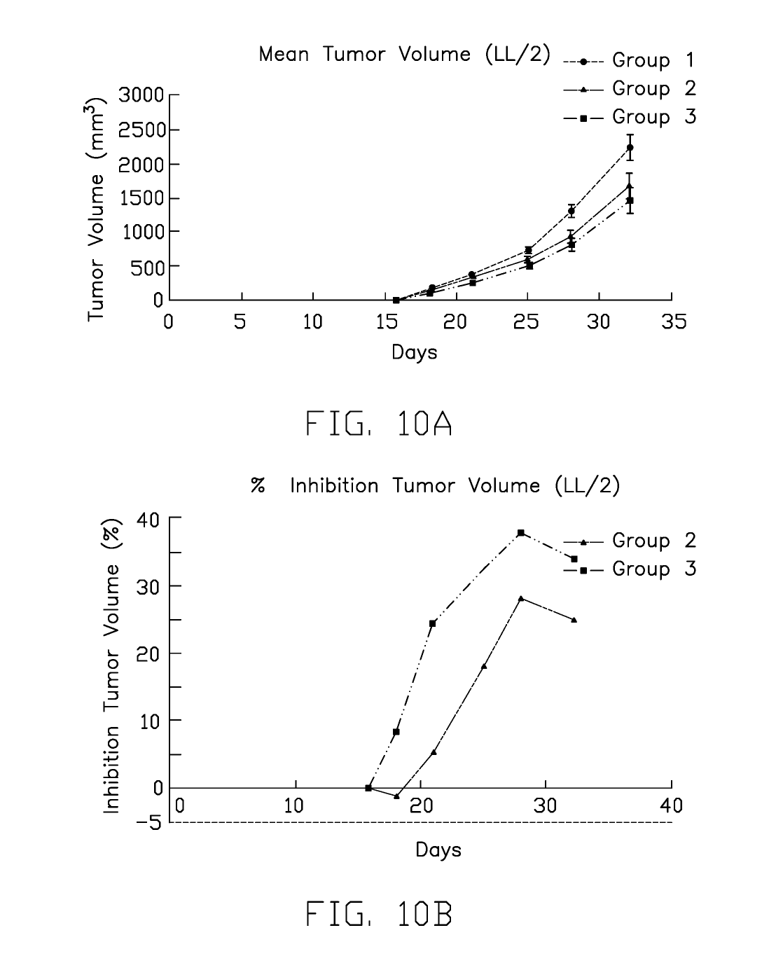

As shown in FIG. 10A, tumor growth curves (i.e., time-dependent change in mean tumor volume) of the three groups show a reduction in mean tumor volume in Group 3. Mean percentage of tumor volume inhibition was also calculated from the measured tumor volume according to the formula: mean % inhibition=(mean(C)−mean(T))/mean(C)×100%; wherein T means current group value, and C means control group value. As shown in FIG. 10B, Group 3 also exhibited greater tumor volume inhibition than Group 2.

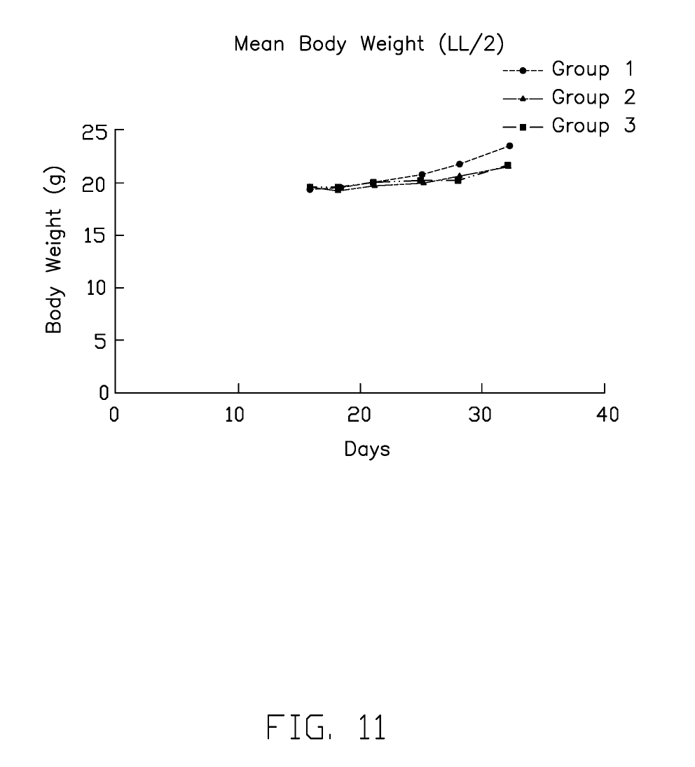

Body weight of the mice was also monitored during the course of the study. As shown in FIG. 11, no adverse effect on body weight was observed in Group 3.

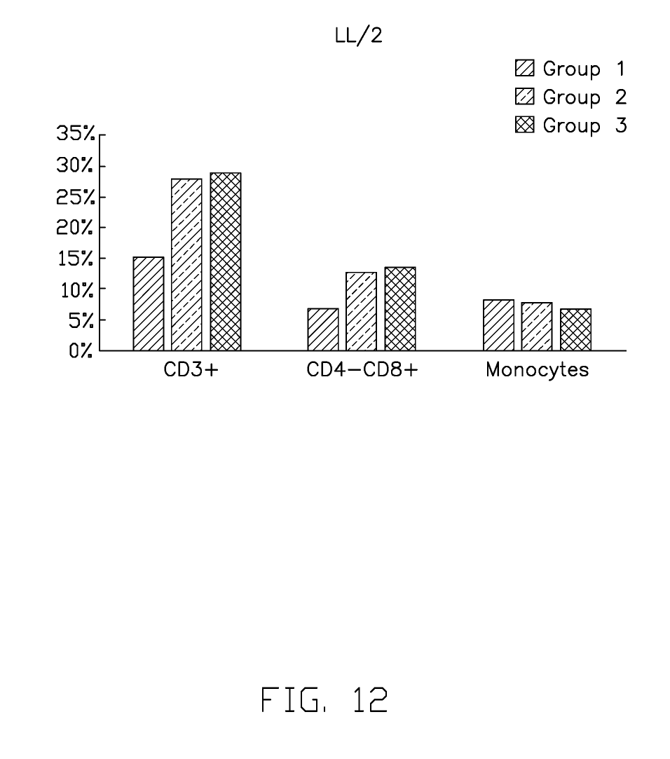

Further, as shown in FIG. 12, flow cytometric analysis of live cells in the tumor revealed that, as compared with the other groups, Group 3 contains higher percentages of CD3+ T cells and CD4−CD8+ T cells and a lower percentage of monocytes over the CD45+ cell population. As the presence of monocytes in the tumor microenvironment has been known to negatively modulate the anti-tumor effects mediated by anti-PD-1 antibody, the results suggest upregulation of accessibility of cytotoxic immune cells and downregulation of accessibility of suppressive immune cells to the tumor microenvironment.

The results shown in FIGS. 10-12 unambiguously demonstrate the synergistic effect of PTX-9908 in anti-PD-1 treatment for lung cancer.

In sum, according to various embodiments of the present disclosure, the peptide having one of SEQ ID Nos. 1-3 (e.g., PTX-9908) is complementary to and synergistic with immunotherapeutics by allowing modulation of tumor immune microenvironment and/or regulation of accessibility of immune cells to the tumor, therefore improving efficacy of the immunotherapy.

Previous descriptions are only embodiments of the present disclosure and are not intended to limit the scope of the present disclosure. Many variations and modifications according to the claims and specification of the disclosure are still within the scope of the claimed disclosure. In addition, each of the embodiments and claims does not have to achieve all the advantages or characteristics disclosed. Moreover, the abstract and the title only serve to facilitate searching patent documents and are not intended in any way to limit the scope of the claimed disclosure.

What is claimed is:

1. A therapeutic combination for treating cancer in a subject having a tumor, comprising:
a peptide comprising one of SEQ ID NOs. 1-3 and being capable of selectively binding to CXC chemokine receptor 4 (CXCR4); and
an immunotherapeutics for treating the cancer.

2. The therapeutic combination according to claim 1, wherein the immunotherapeutics selectively targets CTLA-4, PD-1, PD-L1, TIM-3, LAG-3, B7-1, B7-H3, NKG2A, KIR, BTLA, VISTA/PD-1H, TIGIT, CD96, OX40, CD28, ICOS, HVEM, 41BB, CD40L, CD137, GITR, CD27, CD30, DNAM-1, CD28H or coreceptors thereof.

3. The therapeutic combination according to claim 2, wherein the immunotherapeutics is an antibody, a vaccine, a cytokine, a protein, a peptide, an expression vector encoding the protein or the peptide, a small molecule, an RNAi, or an aptamer.

4. The therapeutic combination according to claim 1, wherein the immunotherapeutics is autologous immune cells, tumor-specific autologous T cells, T-cell receptor (TCR)-engineered T cells, or chimeric antigen receptor T (CAR-T) cells.

5. The therapeutic combination according to claim 1, wherein an immune microenvironment of the tumor is modulated when the peptide binds to CXCR4.

6. The therapeutic combination according to claim 1, wherein accessibility of immune cells to the tumor is regulated when the peptide binds to CXCR4.

7. The therapeutic combination according to claim 6, wherein the immune cells are CD45+ cells, CD3+ T cells, CD4+CD8− T cells, CD4−CD8+ T cells, T-reg cells, NK cells, NKT cells, macrophages, granulocytes, or monocytes.

8. The therapeutic combination according to claim 1, wherein the cancer is breast cancer, colon cancer, lung cancer, pancreatic cancer, prostate cancer, kidney cancer, liver cancer, lymphoma or melanoma.

9. A method for treating cancer in a subject having a tumor, comprising a step of:
administering to the subject a therapeutic combination comprising:
a peptide comprising one of SEQ ID NOs. 1-3 and being capable of selectively binding to CXC chemokine receptor 4 (CXCR4); and
an immunotherapeutics for treating the cancer.

10. The method according to claim 9, wherein the peptide is administered to the subject intravenously, subcutaneously, or intraperitoneally.

11. The method according to claim 9, wherein the immunotherapeutics selectively targets CTLA-4, PD-1, PD-L1, TIM-3, LAG-3, B7-1, B7-H3, NKG2A, KIR, BTLA, VISTA/PD-1H, TIGIT, CD96, OX40, CD28, ICOS, HVEM, 41BB, CD40L, CD137, GITR, CD27, CD30, DNAM-1, CD28H or coreceptors thereof.

12. The method according to claim 11, wherein the immunotherapeutics is an antibody, a vaccine, a cytokine, a protein, a peptide, an expression vector encoding the protein or the peptide, a small molecule, an RNAi, or an aptamer.

13. The method according to claim 9, wherein the immunotherapeutics is autologous immune cells, tumor-specific autologous T cells, T-cell receptor (TCR)-engineered T cells, or chimeric antigen receptor T (CAR-T) cells.

14. The method according to claim 9, wherein an immune microenvironment of the tumor is modulated when the peptide binds to CXCR4.

15. The method according to claim 9, wherein accessibility of immune cells to the tumor is regulated when the peptide binds to CXCR4.

16. The method according to claim 15, wherein the immune cells are CD45+ cells, CD3+ T cells, CD4+CD8− T cells, CD4−CD8+ T cells, T-reg cells, NK cells, NKT cells, macrophages, granulocytes, or monocytes.

17. The method according to claim 9, wherein the cancer is breast cancer, colon cancer, lung cancer, pancreatic cancer, prostate cancer, kidney cancer, liver cancer, lymphoma or melanoma.
